(19) United States
(12) Patent Application Publication
(10) Pub. No.: US 2011/0015452 A1
(43) Pub. Date: Jan. 20, 2011

(54) METHOD FOR PREPARING FLUORINATED COMPOUNDS

(75) Inventors: Michel Devic, Sainte Foy Lee Lyon (FR); Laurent Wendlinger, Soucieu en Jarrest (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: ARKEMA FRANCE, Colombes (FR)

(21) Appl. No.: 12/934,758

(22) PCT Filed: Mar. 27, 2009
(86) PCT No.: PCT/IB2009/005092

§ 371 Date: Sep. 27, 2010

(30) Foreign Application Priority Data
Mar. 28, 2008 (FR) 08 01 729

(51) Int. Cl. C07C 21/18

(52) U.S. Cl. 570/140; 570/156

(57) ABSTRACT

The invention relates to a method for selective dehydrofluorination on a mixed catalyst based on chromium and nickel on a carrier based on aluminium. The invention is used for the synthesis of 2,3,3,3-tetrafluoro-1-propene and 1,2,3,3,3-pentafluoro-1-propene.

### FIELD OF THE INVENTION

The invention concerns a process for the preparation of fluorinated compounds, in particular the fluorinated compounds 1225ye and 1234yf.

### TECHNOLOGICAL BACKGROUND

The hydrofluorocarbons (HFC) and in particular the hydrofluoroolefins, such as 2,3,3,3-tetrafluoro-1-propene (HFO 1234yf) are compounds known for their properties as refrigerants and heat-transfer fluids, extinguishing agents, propellants, foaming agents, swelling agents, gaseous dielectric compounds, polymerization or monomer media, support fluids, abrasive agents, drying agents and fluids for an energy production unit. Unlike CFCs and HCFCs, which are potentially dangerous for the ozone layer, HFOs do not contain chlorine and therefore do not pose a problem for the ozone layer.

Several processes for the manufacture of 1234yf are known.

WO2008/002499 describes a process for the production of a mixture of 2,3,3,3-tetrafluoro-1-propene (HFO 1234yf) and 1,3,3,3-tetrafluoro-1-propene (HFO 1234ze) by pyrolysis of 1,1,1,2,3-pentafluoropropane (HFC 245eb). The temperatures used are described as being from 450° C. to 900° C., and in general from 600° C. to 750° C. which are the temperatures used in the examples. For a conversion of 245eb which is greater than 50%, the ratio of the products 1234yf:1234ze is approximately 1.3-1.4.

WO 2008/002500 describes a process for the production of a mixture of 2,3,3,3-tetrafluoro-1-propene (HFO 1234yf) and 1,3,3,3-tetrafluoro-1-propene (HFO 1234ze) by the catalytic conversion of 1,1,1,2,3-pentafluoropropane (HFC 245eb) on a dehydrofluorination catalyst. The preferred dehydro-fluorination catalyst is fluorinated alumina or aluminium fluoride (aluminium oxyfluoride and fluoride), the catalyst used in the example being aluminium oxyfluoride. Are also described dehydrofluorination catalysts chosen from the oxides, fluorides and oxyfluorides of different metals, among which are chromium and nickel. No example of these catalysts is given. The temperatures used are described as being from 200° C. to 500° C., and in general from 300° C. to 450° C., the higher conversions being obtained at 400° C. and more in the examples. For a conversion of 245eb greater than 50%, the ratio of the products 1234yf/1234ze is approximately 1.9-2.0.

These two aforementioned applications therefore relate to the production of a mixture containing a substantial portion of product 1234ze.

WO 2007/056194 describes the preparation of HFO by conversion of a compound of formula (I) CF₃CHXCH₂X to a compound of formula (II) CF₃CZCHZ, formulae in which X is independently Cl or F, and Z is independently H or F. It is indicated that the selectivity of certain compounds, such as in particular tetrafluoropropene, and more preferentially still HFO-1234yf and/or HFO-1234ze, and at least 70%, preferably at least 80%. The reaction takes place in gas phase, in the presence of a catalyst. The catalysts described in this application are catalysts such as for example FeCl₃, chromium oxyfluoride, Ni (including Ni mesh), NiCl₂, CrF₃, and their mixtures. Other catalysts described are carbon-supported catalysts, antimony based catalysts, aluminium-based catalysts (such as AlF₃ and Al₂O₃), palladium-based, platinum-based, rhodium-based and ruthenium-based catalysts. A preferred embodiment is the preparation of 1234yf by dehydrohalogenation of 245eb, in particular on a nickel-based catalyst, a carbon-based catalyst or a combination of these. The temperature indicated as being appropriate is comprised between 450° C. and 600° C. A conversion of at least 50% and a selectivity of 1234yf of at least 70%, preferably at least 80% and more preferentially of at least 90% is also indicated. Examples 1-16 of WO2007/056194 give several catalysts and temperature conditions for the reaction, with variable conversions and selectivities. Using the same catalyst, the increase in temperature leads to an increase in conversion but accompanied by a concomitant decrease in selectivity, therefore affecting the yield of 1234yf.

WO2008/030440 describes a process comprising a first stage of hydrogenation of 1,2,3,3,3-pentafluoro-1-propene (1225ye) to 1,1,1,2,3-pentafluoropropane (245eb), which is then dehydrohalogenated in order to produce the sought product 2,3,3,3-tetrafluoro-1-propene (1234yf). The catalyst used in the dehydrohalogenation reaction is chosen from the group consisting of aluminium fluoride; gamma alumina; fluorinated alumina; metal on aluminium fluoride, metal on fluorinated alumina; oxides, fluorides and oxyfluorides of magnesium, zinc and mixtures of magnesium and zinc and/or aluminium; lanthanum oxide and fluorinated lanthanum oxide; chromium oxides, chromium oxyfluorides, and cubic chromium trifluoride; carbon, acid-washed carbon, activated carbon, three-dimensional carbonaceous materials; and metal compound supported on carbon; the metal being in the form of oxide, fluoride or oxyfluoride of at least one metal from the group consisting of sodium, potassium, rubidium, caesium, yttrium, lanthanum, cerium, praseodymium, neodymium, samarium, chromium, iron, cobalt, rhodium, nickel, copper, zinc, and their mixtures. The examples mention a mixture of Cr/Co as metal. The conversion obtained is approximately 50%, for a temperature of 250° C. and a contact time of 30 seconds.

The document WO2007/056128 describes the dehydrohalogenation reaction of compounds of formula (I) CF₃CFₙCHₘXₐ₋ₘ to a compound of formula (II) CF₃CZ═CHZ. This reaction is in particular carried out in the presence of a reducing agent, hydrogen.

Similarly, dehydrofluorination reactions of 1,1,1,2,3,3-hexafluoropropane (HFC 236ea) to 1,2,3,3,3-pentafluoropropene-1 (1225ye) are known.

The document U.S. Pat. No. 5,396,000 describes the preparation of 1,1,1,2,3-pentafluoropropane by catalytic dehydrohalogenation of 1,1,1,2,3,3-hexafluoropropane (236ea) to 1,2,3,3,3-pentafluoropropene-1 (1225ye), followed by a hydrogenation in order to produce the sought compound. The dehydrohalogenation of 236ea to 1225ye is carried out in gas phase, on a catalyst based on fluorinated alumina. The selectivity of 1225ye is not indicated.

The document U.S. Pat. No. 5,679,875 describes the preparation of 1,1,1,2,3-pentafluoropropane by catalytic dehydrohalogenation of 1,1,1,2,3,3-hexafluoropropane (236ea) to 1,2,3,3,3-pentafluoropropene-1 (1225ye), followed by a hydrogenation in order to produce the sought compound. The reactions are carried out in gas phase. The dehydrohalogenation catalyst is chromium III oxide or oxyfluoride, or carbon. Oxygen can be added to the stream of 1,1,1,2,3,3-hexafluoropropane to maintain the catalytic activity over time. The selectivities are high, but the conversion is not always very high.

EP-A-974571 describes a dehydrofluorination process of 1,1,1,3,3-pentafluoropropane to 1,1,1,3-tetrafluoro-2-propene.

A need therefore exists for a process for the preparation of 1234yf and 1225ye from 245eb and 236ea respectively, with a high selectivity for a high conversion, and therefore a high yield. A need also exists for a catalytic process having a high productivity.

### SUMMARY OF THE INVENTION

The invention therefore provides a selective dehydrofluorination process on a mixed catalyst based on chromium and nickel on a support based on aluminium.

The invention also provides a process for the dehydrofluorination of a compound of formula (I) CF₃—CHF—CHFX, in which X is hydrogen or fluorine to a compound of formula (II) CF₃—CF═CHX, on a mixed catalyst based on chromium and nickel on a support based on aluminium.

According to the embodiments:
the compound of formula (I) is the compound of formula (Ia) CF₃—CHF—CHF₂ and the compound of formula (II) is the compound of formula (IIa) CF₃—CF═CHF.
the compound of formula (I) is the compound of formula (Ib) CF₃—CHF—CH₂F and the compound of formula (II) is the compound of formula (IIb) CF₃—CF═CH₂.
the mixed catalyst comprises an oxide, a halide or an oxyhalide of chromium in a mixture with an oxide, a halide or an oxyhalide of nickel.
the mixed catalyst comprises a fluoride or an oxyfluoride of chromium in a mixture with a fluoride or an oxyfluoride of nickel.
the support based on aluminium is a halide or an oxyhalide of aluminium.
the support based on aluminium is a fluoride or an oxyfluoride of aluminium.
the catalyst comprises by weight 0.5 to 20% chromium and 0.5 to 20% nickel, these elements being present according to a molar ratio between 0.5 and 5.
the process is implemented in gas phase.
the process is implemented at a temperature comprised between 150° C. and 600° C., preferably between 300 and 500° C. and advantageously between 300 and 400° C.
the process is implemented with a contact time comprised between 0.1 and 100 seconds, preferably between 1 and 50 seconds and advantageously between 2 and 20 seconds.
the process is implemented in the presence of hydrogen, the molar ratio H₂/1,1,1,2,3-pentafluoropropane being comprised between 0.3 and 30, in particular between 0.5 and 20, advantageously between 1 and 10.

The invention also provides a use of a mixed catalyst based on chromium and nickel on a support based on aluminium as a catalyst for a selective dehydrofluorination reaction.

### DETAILED DESCRIPTION OF EMBODIMENTS

The invention uses a mixed catalyst of chromium and nickel.

This mixed catalyst, contains both chromium and nickel. The molar ratio Cr:Ni, with respect to the metallic element, is generally comprised between 0.5 and 5, for example between 0.7 and 2, in particular close to 1. The catalyst can contain by weight 0.5 to 20% chromium and 0.5 to 20% nickel and, preferably, between 2 and 10% each of the metals.

The metal can be present in metallic form or in the form of derivatives, in particular oxide, halide or oxyhalide, these derivatives, in particular halide and oxyhalide, being obtained by activation of the catalytic metal. Although the activation of the metal is not necessary, it is preferred.

The support is based on aluminium. Several possible supports can be mentioned such as alumina, activated alumina or aluminium derivatives. These aluminium derivatives are in particular aluminium halides or oxyhalides, described for example in U.S. Pat. No. 4,902,838, or obtained by the activation process described below.

The catalyst can contain chromium and nickel in a non-activated form or in an activated form, on a support which is also subjected to activation of the metal or not.

The catalyst can be prepared from alumina (in general a so-called activated alumina; this activated alumina is an alumina with high porosity, and is distinct from the alumina that has been subjected to activation treatment of the metal). In a first stage the alumina is converted to aluminium fluoride or to a mixture of aluminium fluoride and alumina, by fluorination using air and hydrofluoric acid, the conversion rate of the alumina to aluminium fluoride essentially depends on the temperature at which the fluorination of the alumina is carried out (in general between 200° C. and 450° C., preferably between 250° C. and 400° C.). The support is then impregnated using aqueous solutions of chromium and nickel salts or using aqueous solutions of chromic acid, a nickel salt and methanol (which serves as a chromium reducing agent). As chromium and nickel salts, chlorides can be used, or other salts such as, for example, nickel oxalates, formates, acetates, nitrates and sulphates or bichromate, in so far as these salts are soluble in the quantity of water capable of being absorbed by the support. The catalyst can also be prepared by direct impregnation of the alumina (which in general is activated) using the solutions of the chromium and nickel compounds mentioned above. In this case, the conversion of at least a portion (for example 70% or more) of the alumina to aluminium fluoride or aluminium oxyfluoride is carried out during the activation stage of the catalyst metal.

The activated aluminas which can be used for the preparation of the catalyst are well-known products, which are available commercially. They are generally prepared by calcination of aluminium hydrates (aluminium hydroxides) at a temperature comprised between 300° C. and 800° C. The aluminas (activated or not) can contain significant levels (up to 1000 ppm) of sodium without this harming the catalytic performances.

Preferably, but without it being necessary, the catalyst is conditioned or activated, i.e. converted to active and stable constituents (under the reaction conditions) by a prior operation called activation.

This treatment can be carried out either “in situ” (in the dehydrofluorination reactor) or in a suitable apparatus designed to withstand the activation conditions.

This activation stage comprises in general the following stages:
A drying stage. This drying stage is carried out at high temperature (250° C. to 450° C., preferably 300° C. to 350° C.) in general under a stream of nitrogen or air. This stage can optionally be preceded in a first phase by a first drying stage at low temperature (100° C. to 150° C., preferably 110° C. to 120° C.) in the presence of air or nitrogen. The duration of the drying stage can be comprised between 1 and 50 hours.
A fluorination stage. This fluorination stage is implemented at low temperature (180° C. to 350° C.) using a mixture of hydrofluoric acid and nitrogen, controlling the HF content so that the temperature does not exceed 350° C. The duration of the fluorination stage can be comprised between 1 and 50 hours.
Optionally a finishing stage under a stream of hydrofluoric acid which is pure or diluted with nitrogen at a temperature which can range up to 450° C. The duration of the finishing stage can be comprised between 1 and 15 hours.

During this operation, the catalytic precursors (for example nickel and chromium halides, nickel chromate or bichromate, chromium oxide) are converted to the corresponding fluorides and/or oxyfluorides, which leads to a release of water and/or hydrochloric acid. Chemical analysis of the elements (chromium, nickel, fluorine, aluminium, oxygen), after this activation, makes it possible to verify the mineral composition of the catalyst.

Such a catalyst is described in EP-A-486333, in particular page 3, lines 11-48, Examples IA, 2A and 4A, passages to which reference is made. This catalyst is used in this document as a fluorination catalyst, in a catalytic fluorination reaction of chloro-1-trifluoro-2,2,2-ethane (133a) with gaseous HF in order to produce tetrafluoro-1,1,1,2-ethane (134a).

The dehydrofluorination reaction is the reaction in which HF is eliminated from a starting compound, leading to the creation of a double bond in the final compound. The reaction under consideration is selective, in the sense that the starting compound (typically a hydrofluorocarbon compound having up to 4 carbon atoms) has a stereochemistry such that the elimination of HF leads to at least two position isomers. The starting fluorocarbon compounds are in particular those comprising a trifluoromethyl terminal group.

The dehydrofluorination reaction under consideration is in particular the dehydrofluorination reaction of a compound of formula (I) CF₃—CHF—CHFX, in which X is hydrogen or fluorine to a compound of formula (II) CF₃—CF═CHX.

According to a first embodiment, the compound of formula (I) is the compound of formula (Ia) CF₃—CHF—CHF₂ (236ea) and the compound of formula (II) is the compound of formula (IIa) CF₃—CF═CHF (1225ye).

According to a second embodiment, the compound of formula (I) is the compound of formula (Ib) CF₃—CHF—CH₂F (245eb) and the compound of formula (II) is the compound of formula (IIb) CF₃—CF═CH₂ (1234yf).

The dehydrofluorination reaction is in general implemented in gas phase.

The catalyst can be present in any appropriate form, for example in the form of a fixed or fluidized bed, preferably in a fixed bed. The direction of flow can be from top to bottom or from bottom to top.

The temperature can be comprised between 150° C. and 600° C., preferably between 300 and 500° C. and advantageously between 300 and 400° C. The pressure can be atmospheric, or less than or greater than this atmospheric pressure.

The contact time (ratio between the volume of catalyst and the total flow of the load) is in general comprised between 0.1 and 100 seconds, preferably between 1 and 50 seconds, in particular for 236ea and advantageously between 2 and 20 seconds, in particular for 245eb.

A diluting gas (nitrogen, helium or argon) can be used in the reaction. Hydrogen can also be injected, for example continuously, or discontinuously. The molar ratio H₂/245eb can vary to a great extent, in particular between 0.3 and 30, in particular between 0.5 and 20, advantageously between 1 and 10.

The reaction is implemented in a reactor dedicated to reactions involving halogens. Such reactors are known to a person skilled in the art, and can comprise internal coatings based for example on Hastelloy®, Inconel®, Monel® or fluoropolymers. The reactor can also comprise heat exchange means, if necessary.

The supply of reagents can in general be carried out continuously, or can be in stages if appropriate.

The product of the reaction, containing in particular the sought 1234yf or 1225ye, HF and possible by-products and starting products that have not reacted, is separated in a standard fashion. The possible reagents that have not reacted are advantageously recycled into the process.

In the process, the conversion is more than 50%, preferably more than 70% and advantageously more than 80%. The selectivity is very high, in general more than 80%, preferably more than 90% and advantageously more than 95%. The yield is in general greater than 80%.

It will be recalled that:
the conversion rate is the % of the starting product that has reacted (number of moles of starting product having reacted/number of moles of starting product introduced);
the selectivity of the sought product is the ratio of the number of moles of sought product formed/number of moles of starting product having reacted;
the yield of sought product is the ratio of the number of moles of sought product formed/number of moles of starting product introduced, the yield of sought product can also be defined as the product of the conversion and the selectivity.
the contact time is the inverse of the space velocity (or Gas Hourly Space Velocity (GHSV))
the space velocity is the ratio between the total volume flow rate and the volume of the catalytic bed, under normal temperature and pressure conditions.

### EXAMPLES

The following examples illustrate the invention without limiting it.

### Example 1

### Preparation of the Catalyst

The catalyst used is an Ni—Cr/AlF₃ catalyst prepared as follows.

343 g of a support obtained in a previous stage by fluorination of GRACE HSA alumina in a fixed bed at about 280° C. using air and hydrofluoric acid (volumic concentration of 5 to 10% of this acid in the air) is placed in a rotary evaporator. The starting GRACE HSA alumina has the following physicochemical characteristics:
form: beads which are 0.5-2 mm in diameter
BET surface area: 220 m²/g
pore volume: 1.3 cm³/g
   Moreover two separate aqueous solutions are prepared:
   (a) chromic solution with nickel chloride added containing:

| | |
|---|---|
| chromic anhydride | 55 g |
| nickel chloride hexahydrate | 130 g |
| water | 63 g |

(b) methanolic solution containing:

| | |
|---|---|
| methanol | 81 g |
| water | 8 g |

These two solutions are introduced simultaneously at a temperature of 40° C. under atmospheric pressure and over approximately 2 hours, onto the support under stirring. After a stage of maturation under nitrogen, the catalyst is dried under nitrogen, then under vacuum at 65° C. then at around 90° C. for 6 hours.

500 g of impregnated solid is loaded into a tubular reactor made of Inconel. The catalyst is firstly dried under nitrogen sweeping at low temperature then up to 320° C., at atmospheric pressure. It is then fluorinated in the presence of a hydrofluoric acid/nitrogen mixture (volumic concentration of 5 to 10% of this acid in nitrogen) at 320° C. then up to 390° C. The supply of HF is then cut off. The nitrogen sweeping is maintained for 15 minutes at 390° C. then the catalyst is cooled down to 60° C. under nitrogen sweeping.

The characteristics of the catalyst after activation are the following:
BET surface area: 40 m²/g
pore volume: 0.4 cm³/g
chemical composition by weight:
   Al: 25%
   F:58%
   Cr: 5.3%
   Ni: 6.4%

### Example 2

### Dehydrofluorination of 245eb

A reactor with a volume of 50 cm³ and an inside diameter of 2.1 cm is used, containing 20 g of the catalyst of Example 1 in the form of a fixed bed with a height of 6.5 cm. The pressure is one atmosphere. The reagent is mixed with hydrogen and injected into the reactor, which has been heated beforehand to the reaction temperature. A sample of the gaseous effluent is then analyzed by gas chromatography.

The following results are then obtained (MR means molar ratio).

| | | MR | | Selec- | Selec- | Productivity |
|---|---|---|---|---|---|---|
| | Contact | H₂/ | Conver- | tivity | tivity | 1234yf |
| Temp. | time | 245eb | sion | 1234yf | 1234ze | Kg/h/m³ cata |
| 377° | 8.8 sec | 1.4 | 99.8% | 89.6% | 9.1% | 773 |

It should be noted that the productivity with the mixed catalyst is very high, whilst ensuring a conversion and a selectivity which are also very high.

### Example 3

### Dehydrofluorination of 245eb to 1234yf

A reactor with a volume of 25 cm³ is used containing 10 g of the catalyst of Example 1 in the form of a fixed bed. The pressure is one atmosphere.

The following results are then obtained (MR means molar ratio).

| | | MR | | Selec- | Selec- | Productivity |
|---|---|---|---|---|---|---|
| | Contact | H₂/ | Conver- | tivity | tivity | 1234yf |
| Temp. | time | 245eb | sion | 1234yf | 1234ze | Kg/h/m³ cata |
| 373° | 8.9 sec | 3 | 87% | 90% | 9% | 436 |

It should be noted that the productivity with the mixed catalyst is very high, whilst ensuring a conversion and a selectivity which are also very high.

### Example 4

### Dehydrofluorination of 236ea to 1225ye

The same reactor loaded with the same quantity of catalyst as for Example 2 is used. The pressure is 1 atmosphere. The following results are then obtained (MR means molar ratio).

| | | MR | | Selec- | Productivity |
|---|---|---|---|---|---|
| | Contact | H₂/ | Conver- | tivity | 1234ye |
| Temp. | time | 236ea | sion | 1225ye | Kg/h/m³ cata |
| 375° | 10.4 sec | 2.14 | 50.1% | 97.6% | 3.19 |

The same results are also obtained at the end of 100 hours of operation.

1. Selective dehydrofluorination process on a mixed catalyst based on chromium and nickel on a support based on aluminium.

2. Dehydrofluorination process according to claim 1 of a compound of formula (I) CF₃—CHP—CHFX, in which X is hydrogen or fluorine to a compound of formula (II) CF₃—CF═CHX, on a mixed catalyst based on chromium and nickel on a support based on aluminium.

3. Process according to claim 2, in which the compound of formula (I) is the compound of formula (Ia) CF₃—CHF—CHF₂ and the compound of formula (II) is the compound of formula (IIa) CF₃—CF═CHF.

4. Process according to claim 3, in which the compound of formula (I) is the compound of formula (Ib) CF₃—CHF—CH₂F and the compound of formula (II) is the compound of formula (IIb) CF₃—CF═CH₂.

5. Process according to claim 1, in which the mixed catalyst comprises a chromium oxide, halide or oxyhalide in a mixture with a nickel oxide, halide or oxyhalide.

6. Process according to claim 1, in which the mixed catalyst comprises a chromium fluoride or oxyfluoride in a mixture with a nickel fluoride or oxyfluoride.

7. Process according to claim 1, in which the support based on aluminium is an aluminium halide or oxyhalide.

8. Process according to claim 1, in which the support based on aluminium is an aluminium fluoride or oxyfluoride.

9. Process according to claim 1, in which the catalyst comprises by weight 0.5 a 20% chromium and 0.5 to 20% nickel, these elements being present according to a molar ratio between 0.5 and 5.

10. Process according to claim 1, implemented in gas phase.

11. Process according to claim 1, implemented at a temperature comprised between 150° C. and 600° C., preferably between 300 and 500° C. and advantageously between 300 and 400° C.

12. Process according to claim 1, implemented with a contact time comprised between 0.1 and 100 seconds, preferably between 1 and 50 seconds and advantageously between 2 and 20 seconds.

13. Process according to claim 1, implemented in the presence of hydrogen, the molar ratio H₂/1,1,1,2,3-pentafluoropropane being comprised between 0.3 and 30, in particular between 0.5 and 20, advantageously between 1 and 10.

14. A method of performing a selective dehydrofluorination reaction comprising conducting dehydrofluorination in the presence of a mixed catalyst based on chromium and nickel on a support based on aluminium.
